(19) United States
(12) Patent Application Publication
(10) Pub. No.: US 2021/0069035 A1
(43) Pub. Date: Mar. 11, 2021

(54) SEAMLESS ELASTIC APPENDAGE COVER

(71) Applicant: JUDITH COTTRELL, HOUSTON, TX (US)

(72) Inventor: JUDITH COTTRELL, HOUSTON, TX (US)

(21) Appl. No.: 17/017,349

(22) Filed: Sep. 10, 2020

Related U.S. Application Data
(60) Provisional application No. 62/898,352, filed on Sep. 10, 2019.

(51) Int. Cl. A61F 15/00

(52) U.S. Cl. CPC A61F 15/004; A61F 15/008

(57) ABSTRACT

An appendage cover assembly includes cover 101 having a body that forms a cavity. The cover body is seamless and waterproof and has an upper portion adjacent an open end, a tip portion adjacent a distal end, and a central portion between the upper portion and the tip portion. The upper portion of the cover body is configured to selectively expand and contract in size larger and smaller than the size as compared to the other portions. The upper portion is configured to secure the cover to the appendage and/or bandage. The upper portion compresses onto the bandage away from the wound. The central portion avoids compression on the wound.

### CROSS REFERENCE TO RELATED APPLICATIONS:

This application claims the benefit of earlier filing date and right of priority to U.S. Provisional Application No. 62/898,352, filed 10 Sep. 2019, the contents of which is incorporated by reference herein in its entirety.

### BACKGROUND OF THE INVENTION

### 1. Field of the Invention

The present application relates to protective appendage covers, and more particularly to protective covers that prevent a bandage or a medically treated site from contamination from environmental elements.

### 2. Description of Related Art

An injury on a limb sometimes require a bandage to aid in healing the injury. Bandage materials can be, but are not limited to, a generic piece of cloth, an adhesive bandage, a cotton roll bandage, a compression bandage, or an orthopedic cast. In some instances, the injury may require an additional protective sheath to cover the bandage in order to prevent liquids and debris from contaminating the bandage.

For instance, a dog having an injured limb that required surgical intervention is given generally some type of bandage to cover and support the surgical site post operation. However, the dog may desire to utilize or lick the injured limb which can contaminate the bandage, which may lead to further medical complications at the site of injury. Thus, a protective sheath can be employed to protect the bandage from external contamination. A variety of protective sheaths to protect the bandage have been proposed for injured appendages, however, the proposed sheaths have several drawbacks.

Some appendage covers are made of materials that are not waterproof which allow for liquid based contaminates to permeate through the cover and enter the bandage. Other appendage covers use a waterproof material, but the formation of the cover includes a seam that runs lengthwise along the cover or about its bottom end. Seams for a protective cover present a mode of failure since the seam may fail during manufacturing or during use, thus permitting external contaminants to enter within the cover. Furthermore, having a seam running along the protective cover presents a source of physical irritation for the user as the seam may protrude outward from the cover and catch on objects in passing, or the seam is oriented in a fashion that is noticeable by the user when external pressure is applied to the protective cover. In another proposed cover, the cover is composed entirely of an elastic material, however, the elastic material adds unwanted pressure to the covered portion of the injured limb, including added pressure to local injuries or medical treatment on the limb.

Although strides have been made to improve a protective sheath for an injured limb, some shortcomings remain. It is desired to make a seamless and waterproof bandage cover that does not add unwanted pressure to local injuries on an appendage.

### BRIEF SUMMARY OF THE INVENTION

It is an object of the present application to provide a seamless and waterproof appendage cover configured to overlap a portion of a bandage covering a wound on a user. The appendage cover is configured to prevent the transmission of contaminants into the bandage. The cover may include one or more materials having different material characteristics that may be optionally layered over one another to enhance cover performance.

It is a further object of the present application that the cover avoids applying pressure to the wound area on the user while using pressure to secure it to the bandage about the wound. This is accomplished by the inclusion of different materials into the cover. It is desired that the cover includes different portions having varied levels of compressibility or flexibility, wherein the inclusion of additional materials in the cover help to distinguish compressible portions. Portions of greater compressibility may be used to secure the cover to the bandage while areas of minimal compressibility are located around the wound.

It is understood that the cover may employ a pleated portion having an elastic band located at a cover opening that secures the cover to the bandage and forms a seal against the appendage. In further embodiments, the pleated portion includes bandage clips that help secure the pleated portion of the cover to a bandage on the appendage, thereby resisting the appendage cover from coming off the appendage. Additional details of the assembly are described herein.

Ultimately the invention may take many embodiments. In these ways, the present invention overcomes the disadvantages inherent in the prior art. The more important features have thus been outlined in order that the more detailed description that follows may be better understood and to ensure that the present contribution to the art is appreciated. Additional features will be described hereinafter and will form the subject matter of the claims that follow.

Many objects of the present application will appear from the following description and appended claims, reference being made to the accompanying drawings forming a part of this specification wherein like reference characters designate corresponding parts in the several views.

Before explaining at least one embodiment of the present invention in detail, it is to be understood that the embodiments are not limited in its application to the details of construction and the arrangements of the components set forth in the following description or illustrated in the drawings. The embodiments are capable of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the various purposes of the present design. It is important, therefore, that the claims be regarded as including such equivalent constructions insofar as they do not depart from the spirit and scope of the present application.

### BRIEF DESCRIPTION OF THE DRAWINGS

The novel features believed characteristic of the application are set forth in the appended claims. However, the application itself, as well as a preferred mode of use, and further objectives and advantages thereof, will best be understood by reference to the following detailed description when read in conjunction with the accompanying drawings, wherein:

FIG. 1 is a side view of an appendage cover in accordance with an embodiment of the present application.

FIG. 2 is a side view of the appendage cover of FIG. 1 with an optional treatment to an upper portion.

FIG. 3 is a side view of the appendage cover of FIG. 1 with an optional treatment to an upper portion.

FIG. 4 is a side view of the appendage cover of FIG. 1 with an optional treatment to an upper portion.

FIG. 5 is a cross section of the appendage cover of FIG. 4.

FIG. 6 is a side view of an appendage cover of FIG. 1 with optional treatments from FIGS. 2-4.

While the embodiments and method of the present application is susceptible to various modifications and alternative forms, specific embodiments thereof have been shown by way of example in the drawings and are herein described in detail. It should be understood, however, that the description herein of specific embodiments is not intended to limit the application to the particular embodiment disclosed, but on the contrary, the intention is to cover all modifications, equivalents, and alternatives falling within the spirit and scope of the process of the present application as defined by the appended claims.

### DETAILED DESCRIPTION OF THE INVENTION

Illustrative embodiments of the preferred embodiment are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms to describe a spatial relationship between various components or to describe the spatial orientation of aspects of such components should be understood to describe a relative relationship between the components or a spatial orientation of aspects of such components, respectively, as the embodiments described herein may be oriented in any desired direction.

Embodiments of the present invention overcomes one or more of the above-discussed problems commonly associated with appendage covers. In particular, the system of the present invention is an appendage cover having a seamless and waterproof member that covers bandages (or portions thereof) associated with a local injury/wound on an appendage of a user. The appendage cover may come in a variety of sizes to accommodate appendages (i.e., legs or arms) of humans or animals (e.g., cats, dogs, etc.). Furthermore, the appendage cover is configured to provide an upper portion, central portion, and tip portion. The upper portion is self-tightening to selectively expand and contract in size so as to apply a level of pressure onto the appendage and/or bandage to secure the appendage cover around the wound. One or more additional optional treatments are available to increase abrasion resistance or grip of the appendage cover.

The system and method will be understood, both as to its structure and operation, from the accompanying drawings, taken in conjunction with the accompanying description. Several embodiments of the system may be presented herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless otherwise described.

The system and method of the present application is illustrated in the associated drawings. As used herein, “system” and “assembly” are used interchangeably. It should be noted that the articles “a”, “an”, and “the”, as used in this specification, include plural referents unless the content clearly dictates otherwise. As used herein, “appendage” and “limb” are used interchangeably. As used herein, “bandaging” is a piece of material that either secures a medical device such as a medical dressing or splint, or is itself used to restrict the movement of an appendage of a user. For example, when paired with a dressing, the dressing is applied directly to an open wound, and a bandage is used to hold the dressing in place. In another example, bandages without dressings are used to reduce swelling or provide support to soft tissue injury, bone fractures or to slow blood flow. Bandaging can be, but is not limited to, a generic piece of cloth, an adhesive bandage, a gauze roller bandage, a compression bandage, or an orthopedic cast. In some embodiments, the bandage is a multilayered wrap.

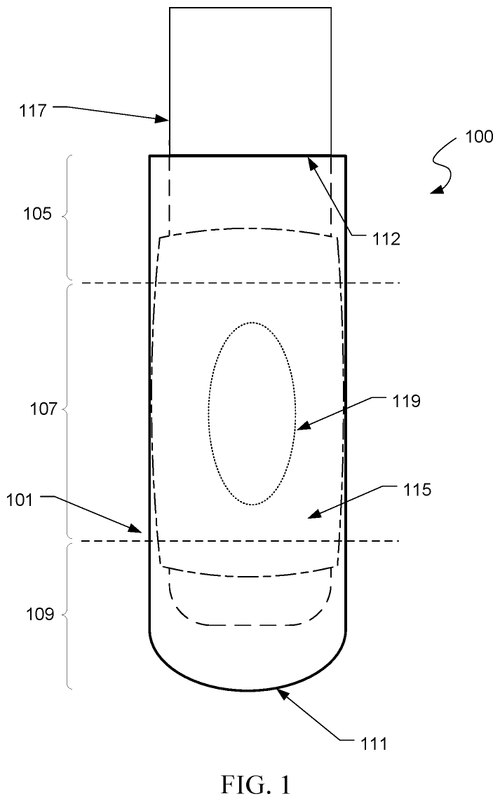

Referring now to the drawings wherein like reference characters identify corresponding or similar elements in form and function through the several views. Referring now to FIG. 1, a side view of appendage cover assembly 100 is illustrated in accordance with an embodiment of the present invention. Assembly 100 is configured to overlay a wound or injury on an appendage of a user/animal and provide protection from contaminants. Assembly 100 includes a cover 101 that is seamless in construction and waterproof.

As seen in FIG. 1, cover 101 is situated over an appendage 117 of a user/animal so as to overlap a wound 119 and corresponding bandage 115. Bandage 115 is wrapped around wound 119 but is typically breathable and is susceptible to contamination. Cover 101 surrounds a portion of bandage 115 and covers wound 119. Cover 101 is distinguishable between at least 3 portions, namely an upper portion 105, a central portion 107, and a tip portion 109.

Assembly 100 includes cover 101 having a body that forms a cavity. The cover body is seamless and has an upper portion 105 adjacent an open end 112, a tip portion 109 adjacent a distal end 111, and a central portion 107 between the upper portion 105 and the tip portion 109. The upper portion 105 of the cover body is configured to selectively expand and contract in size larger and smaller than the size of at least one of the central portion 107 and the tip portion 109. Upper portion 105 is configured to secure cover 101 to appendage 117 and/or bandage 115.

Cover 101 may be made from one or more materials that are layered across its internal and/or external surfaces, however, these are done in a manner so as not to create a seam along its body length as seams are entrance points for contaminants and can adversely affect the wound. It is noted that the materials selected for construction of cover 101 can include surface textures to increase grip or friction on any of the internal cover surface or external cover surface. Portions 105/107/109 may be made from the same materials or different materials. Wherein more than two materials are used, the central materials may differ between the portions without generation of a seam in the outer and inner materials.

For example, known suitable materials may be a single layer of plastic that includes a thermoplastic elastomer with mesh embedded at upper portion 105 to permit a compressive force about the appendage 117. A single layer of plastic may be used and include sheet plastic with elastic threading similar to how diaper straps. Any number of methods or materials may be used, including without limitation to heat molds, injection molds, compression molds, elastic and plastics, special threading patterns for cover 101. In one embodiment, appendage cover 101 is a disposable device made of disposable materials. In alternate embodiments, appendage cover 101 is a reusable device made of durable materials. For example, cover 101 can be a plastic nonporous material.

In this embodiment, the cover (i.e., cover 101) is a body formed as a single bulbous cover having a distal end (i.e., distal end 111) and an open end (i.e., open end 112) wherein the cover forms a cavity for placing the cover around an appendage 117 of a user via the open end. In this embodiment, distal end 111 is an enclosed end of the cover. Cover 101 is manufactured such that the cavity for covering the appendage is formed without a seam running from the open end to the distal end of the cover, nor is there a seam located along the distal end of the cover. Cover 101 is made of a waterproof material that prevents liquids and/or debris from permeating through cover 101.

Depending on the construction of the cover body, different methods of treatments may be necessary. As noted above, assembly 100 may be configured to receive additional and optional treatments to assist in facilitating its purpose. Each optional treatment may be used individually. Alternatively, any of the optional treatments may be used collectively together or in any combination thereof. Particular optional treatments will be described in individual Figures for clarity.

A purpose of assembly 100 is to secure cover body 101 over bandage 115. The size of open end 112 may expand and contract in size (i.e. flexible) in size larger and smaller than the internal diameter/size of central portion 107. Upper portion 105 may include items having elastic characteristics with one or more fasteners, members, bands, and so forth to facilitate this function. Upper portion 105 is the main way of securing the cover over a bandage 115. Upper portion 105 has to cover a larger surface area to avoid unwanted pressure on appendage 117. For example the items may be spaced out to equal 1.5″ or 3″ depending on size of cover. This will also provide better security on the bandage. It is also more convenient and easier to apply on bandage than tie on methods. These elastic characteristics may be situated in between layers of material at upper section 105 or along an internal or external surface of the cover body.

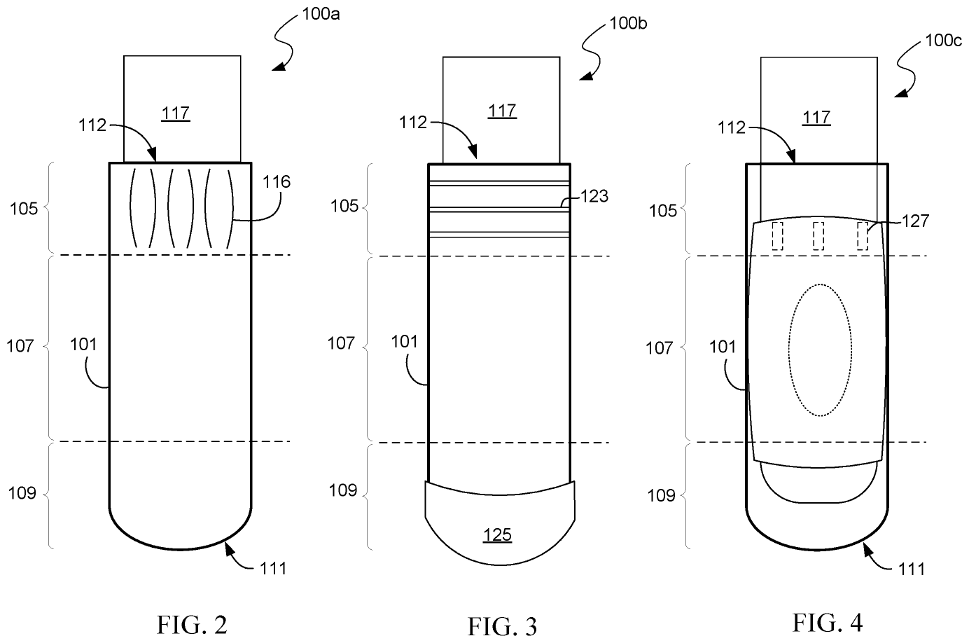

Referring now also to FIG. 2 in the drawings, a side view of assembly 100a is illustrated with a pleat optional treatment. This is similar in form and function to that of assembly 100 except in that it is used to show the pleat optional treatment. Cover 101 cannot be constructed with cut pieces of material that are sewn or adhered together with seams. This is especially true along the base or distal end 111 and the sides of the cover body. Whole or one piece of materials are preferred and should be used to eliminate seams. Using full pieces of material vs multiple cut pieces means having to manage or deal with the extra material.

In this embodiment, cover body 101 is a tubular member having distal end 111 and open end 112. Cover 101 is made from a full piece of material and due to its tubular nature. This may lead to folds or pleats 116 in the cover body. This is especially true at upper portion 105, opposite from distal end 111. Pleats 116 may be formed in any portion 105/107/109 and is formed by the overlaying of the cover body onto itself. The layers may be held together with fasteners such as adhesive, stretchable adhesive, elastic bands, or elastic threading to name a few, so as to permit the expansion and contraction in size or upper portion 105 to openings larger and smaller than that of central portion 107.

Upper portion 105 is configured to induce a compressive force around appendage 117 so as to secure the location or position of assembly 100 relative to bandage 115 and wound 119. Different fastening methods may be used with pleats 116 to induce this selective compressive force in allowing the expansion and contraction in size of open end 112.

Referring now also to FIG. 3 in the drawings, an additional side view of assembly 100 is shown, as assembly 100b. FIG. 3 illustrates assembly 100b in that the optional treatment included to assist in inducing the compressive force is a band 123. Bands 123 are ideally elastic bands allowed to stretch and contract. Bands 123 may be located in any surface or layer of cover 101. It is noted that the surface of cover 101 being internal within the cavity is an internal surface while the surface outside of the cavity is an external surface of the cover 101. Bands 123 may be on internal surfaces, external surfaces or between surfaces of cover 101. Bands 123 may be fastened or secured to cover 101 with any type of known fastener, including stretchable adhesive. Bands 123 may be used in conjunction with that of pleats 116.

In one embodiment, band 123 runs circumferentially along pleat 116 or around upper portion 105 of cover 101. In one embodiment, band 123 is a tubular elastic member having a first diameter when the elastic of band 123 is at a relaxed state and having a second diameter when the elastic of band 123 is at a maximum stretched state. In this embodiment, when band 123 is stretched to a diameter between the first diameter and the second diameter, band 123 is under spring tension to return to the first diameter, wherein the spring tension of band 123 is used to secure appendage cover 100 to an appendage of a user. A variety of widths for band 123 is contemplated to comfortably distribute spring forces against the appendage. In other words, wider widths for band 123 spread out the spring forces of band 123 against an appendage, thereby reducing uncomfortable pressure against the appendage.

The combination of pleat 116, band 123, and cover 101 is an appendage cover having an elastic end proximate to end 112 that secures the appendage cover to an appendage while cover 101 loosely covers a portion of bandage associated with a local injury on an appendage (i.e., cover 101 covers the injury without putting unwanted pressure on the injury).

A cap 125 is also optionally included and is shown in FIG. 3. Cap 125 is located in tip portion 109 and is coupled to the outer surface of cover 101. Cap 125 may be a hardened member to provide protection to a portion of appendage 117. Additionally, cap 125 may include a surface texture or coating to increase grip or abrasion resistance. It is understood that cap 125 may extend into central portion 107 if desired. Additionally, any textures or coatings along surfaces of cap 125 and cover 101 may be included. The texture, coatings, and material characteristics between the internal surface and the external surfaces may vary.

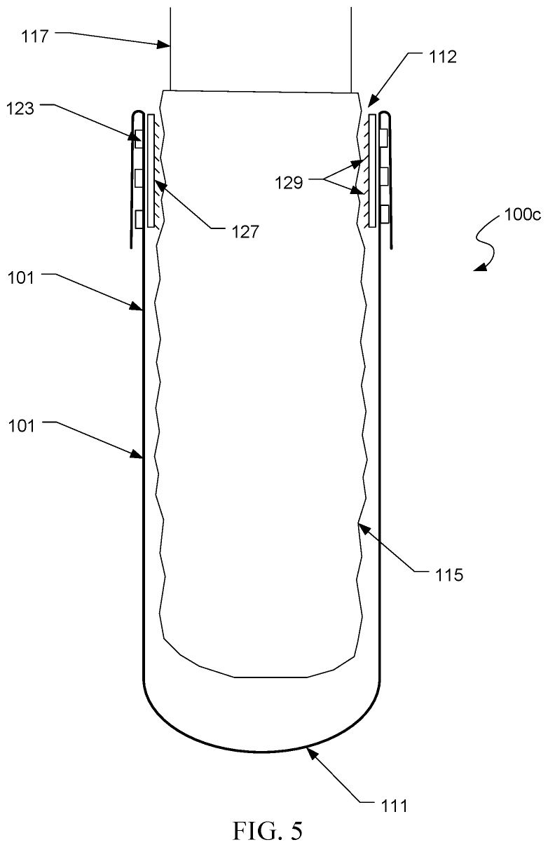

Referring now also to FIGS. 4 and 5 in the drawings, an additional optional side view of assembly 100 is shown, as assembly 100c wherein clips 127 are used. FIG. 4 illustrates assembly 100c which is similar in form and function to that of assembly 100 except in that the optional treatment of a clip 127 is included to assist in securing the assembly to the bandage 117.

In one embodiment, cover 101 has a plurality of clips 127. In this embodiment, clips 127 are in communication with an inner face of pleat 116 or the cover material, wherein the clips 127 have hook members 129 that latch on to bandage 115 associated with appendage 117. In this embodiment, the hook members 129 associated with clips 127 are configured to prevent appendage cover 101 from sliding off the appendage by hooking into the bandage 115 in conjunction with the spring force provided by band 123. For example, the hook members 129 splay outward from clips 127 and/or downward towards distal end 111 with the spring force of band 123 applying pressure to clips 127 against the bandage 115 thereby forcing the hook members 129 into the bandage 115 to prevent appendage cover 101 from slipping.

In one embodiment, clips 127 are elongated members that are oriented lengthwise along appendage cover 101 and are spaced a predetermined distance apart from each adjacent clip such that clips 127 permit pleat 116 and band 123 to stretch between a first diameter and a second diameter.

In FIG. 5 the material of cover 101 is shown folded over on itself in upper portion 105 so as to add an additional layer. This is shown to cover bands 123. Clips 127 are located internally along an internal surface of the cavity. Hook members 129 engage bandage 115. It is understood that where multiple layers are used for the cover body, the inner most layer of material can be longer so that it can be folded down over upper portion 105. This helps to cover, secure, and strengthen the material at the upper portion 105.

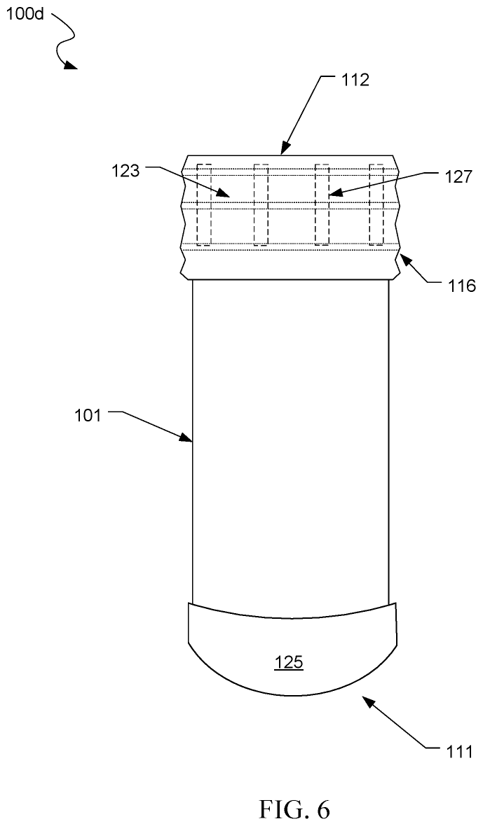

Referring now also to FIG. 6 in the drawings, a combined view of assembly 100 (see assembly 100d) shown with the optional treatments of FIGS. 2-5 are illustrated.

It is understood that any surface of cover 101 or other optional item may include an Anti-slip texture for security. This will “grip” onto bandage or create resistance between cover and bandage material and help prevent slipping down. Texture surfaces created on inside layers of material at upper portion 105 are ideal as this is where the compressive forces are generated to secure assembly 100. Textures may be created with tacky/rubbery substance applied to any surface. These may be sprayed on or directly applied to create the desired textures. Pattern of texture may include one or more ridges or bumps. These may be located on external cover surfaces permanent to cover 101 or may be seen as disposable layers that are discardable. Durable fabrics may be used with mesh or other.

Various materials have been spoken of in this disclosure. Cover 101 has been noted as having one or more material layers, each of which may be different or the same between them. It should be understood that the materials are non rigid in nature and are typically flexible in nature to allow for the expansion and contraction in upper portion 105. Exemplary suitable fabrics may be from the list of: cotton, polyester, nylon, spandex, rayon, mesh fabric, mesh tubing, plastics, thermo-plastic elastomers, polymers, and so forth. Or it may include a type of combined fabric such as thermally bonded PLU or thermally laminated PUL fabric using TPU film.

Assembly 100 may be assembled as a reusable device or as a disposable device. When disposable, cover 101 may be made from a non rigid plastic sheet/roll. For example, it may be a low density polyethylene (LDPE). The thickness may be 0.7 mm or thicker for each layer. With respect to the reusable version, a plastic inner layer(s) with durable material on outer layer(s) is ideal when considering a multilayer cover. Basically, the disposable version may be used as the inside layer and then durable material is applied as the outer layer. It is important though, irrespective of the version (reusable or disposable) that the cover be waterproof.

The particular embodiments disclosed above are illustrative only, as the application may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. It is apparent that an application with significant advantages has been described and illustrated. Although the present application is shown in a limited number of forms, it is not limited to just these forms, but is amenable to various changes and modifications without departing from the spirit thereof.

What is claimed is:

1. An appendage cover for a wound on a user, comprising:
a cover having a body forming a cavity, the body having an open end and a closed distal end, the cover body being seamless, the cover body having:
an upper portion adjacent the open end;
a tip portion adjacent the distal end; and
a central portion between the upper portion and the tip portion;
wherein the upper portion of the cover body is configured to selectively expand and contract in size larger and smaller than the size of at least one of the central portion and the tip portion.

2. The appendage cover of claim 1, wherein the upper portion induces a compressive force upon the user.

3. The appendage cover of claim 1, wherein the upper portion includes an elastic fastener.

4. The appendage cover of claim 1, wherein the cover body is formed from a whole piece of material.

5. The appendage cover of claim 1, wherein the cover body includes a pleat.

6. The appendage cover of claim 5, wherein the pleat is isolated to the upper portion.

7. The appendage cover of claim 1, wherein the cover body includes a band.

8. The appendage cover of claim 7, wherein the band is located in the upper portion.

9. The appendage cover of claim 1, wherein the cover body includes a clip.

10. The appendage cover of claim 9, wherein the clip is located in the upper portion.

11. The appendage cover of claim 1, wherein the central portion avoids a compressive force to a bandage on the user.

12. The appendage cover of claim 1, further comprising:
a cap on the tip portion, the cap in communication with an outer surface of the cover body and configured to protect an appendage of the user.

13. The appendage cover of claim 1, wherein the cover body is formed from a whole piece of material, the cover body formed to include a pleat, the upper portion including a band to induce a compressive force in the open end.

14. The appendage cover of claim 1, wherein the cover body is formed from a whole piece of material, the cover body formed to include a pleat, the upper portion including a clip configured to engage into a bandage and secure the location of the cover body relative to the user.

15. The appendage cover of claim 1, wherein the upper portion includes a band to induce a compressive force in the open end and the upper portion includes a band to induce a compressive force in the open end.

16. The appendage cover of claim 1, wherein the cover body is formed from a whole piece of material, the cover body formed to include a pleat, the upper portion including a band to induce a compressive force in the open end and the upper portion including a band to induce a compressive force in the open end.

17. The appendage cover of claim 1, further comprising:
a cap on the tip portion, the cap in communication with an outer surface of the cover body and configured to protect an appendage of the user.
